(19) United States
(12) Patent Application Publication
(10) Pub. No.: US 2021/0386937 A1
(43) Pub. Date: Dec. 16, 2021

(54) SYRINGE FOR RECONSTITUTING AND INJECTING A PHARMACEUTICAL SOLUTION

(71) Applicant: OROFINO PHARMACEUTICALS GROUP SRL, Roma (IT)

(72) Inventor: Ernesto OROFINO, Roma (IT)

(21) Appl. No.: 17/282,542

(22) PCT Filed: Jul. 24, 2019
(86) PCT No.: PCT/IB2019/056316

§ 371 Date: Apr. 2, 2021

(30) Foreign Application Priority Data
Oct. 5, 2018 (IT) 102018000009185

(51) Int. Cl. A61M 5/315; A61M 5/19; A61M 5/178; A61M 5/31; A61K 31/545; A61K 31/43

(52) U.S. Cl. CPC A61M 5/31596; A61M 5/19; A61M 5/1782; A61M 2005/3128; A61M 5/315; A61K 31/545; A61K 31/43; A61M 5/3134

(57) ABSTRACT

A prefilled syringe includes a tubular containment body extending along an axis between a front end element and a rear end element, a fixed plug and two sliding plugs. A bypass channel in the initial position of the syringe is between the first two plugs which form, together with a third plug at the end of the syringe plunger, a first containment chamber containing a first liquid substance and a second containment chamber containing a second solid or liquid substance. The fixed plug is in contact with the front end element and is provided with a non-through cavity offset with respect to the axis and facing towards the front end element.

The present invention relates to the technical field of syringes for injecting pharmaceutical products, in particular to prefilled multiple chamber syringes.

### BACKGROUND ART

Currently, sterile raw materials in powder form are mainly packed by active ingredient manufacturers either in aluminum containers or in plastic bags, generally made of polyethylene.

In order to be able to market them, manufacturers have demonstrated that each raw material, in addition to maintaining sterility, remains stable in the containers or bags used for a given period of time; in other words, such powders do not degrade for a predefined number of years when stored in such containers or bags.

The sterile raw materials are sold to the manufacturers of the finished pharmaceutical product, who fractionate them using known technologies, marketing the finished product in sterile powder form contained in a bottle. The bottle is accompanied by a glass vial containing the solvent.

The use of high-quality materials for the vial (glass) and of compatible materials for the sterile powder and the use of disposable syringes make this system very costly as a whole.

The use of syringes provided with a compartment for holding a cartridge, made of flexible material, containing pharmaceutical substances kept separate until the moment of use by a special soft septum, is known in the sector. An example of such devices is described in patent US2001/0047162. However, such known devices have some drawbacks. Indeed, due to accumulations and stagnations of the mixture or of the pharmaceutical substance in the corners of the cartridge, not the whole dose is delivered. This makes the dosage of the substance actually injected inaccurate and unreliable.

Other solutions, such as the Debiopass™ by Debiotech S.A. shown in FIG. 1, use a multi-chamber syringe 100, in which the chambers are defined by rubber plugs 140, 160 sliding in a transparent body 120. The initial configuration of the syringe 100 is shown in view (a), in which a chamber between the two plugs 140,160 is already filled with a first liquid 150. The plug 140 is at limit stop position in the body 120. The spout 135 of the syringe supports a needle 130, which is inserted into a vial 200 to aspirate a second liquid 170, visible in configuration (b). While it is being drawn, the second liquid creates the second chamber, pushing back the plug 140 and therefore also the plug 160. The plunger 110 is thus in the maximum protrusion position from the body 120. By removing the needle 130 from the vial 200, the syringe can be used in free state in (c) to administer the two liquids to a patient. The administration of the liquid 170 adjacent to the spout 135 leads to the state in view (d), in which the plug 140 is now in end stop position. By pushing the piston 110 further, the first liquid 150 is injected until the plug 160 comes into contact with the plug 140. In this manner, two liquids were successively injected into the patient with a single injection.

FIG. 2 shows a known plug to be used in the multi-chamber (dynamic) syringe in FIG. 1, in particular as plug 140. A (non-through) cavity 145 which is initially filled with air can be noted. The liquid, while pushing the plunger 110, passes outside the plug and presses it in the direction indicated by the arrow in the figure, because of the presence of the cavity 145 which offers less resistance than the full zone. This creates the side passage for the fluid which is consequently dispensed. Such a plug allows the liquid not to be in initial contact with the spout 135.

A further solution is shown in FIG. 3, taken from document WO2017216651 A1 by the Applicant, which, unlike the solution in FIG. 1, does not draw a liquid, but instead reconstitutes two liquids or a liquid and a powder previously introduced in respective chambers defined by movable plugs 6,7,16, to obtain an injectable solution in a single chamber. For such a purpose, there is a side bypass duct 10, which in the initial state of use of the syringe is located beyond the movable plugs. Furthermore, at the front end 2 there is a liquid storage cavity 20,32 of limited capacity. The liquid in excess is expelled before being able to use the syringe by injecting the solution of the storage cavity. For this reason, the plugs used in this syringe must all be movable and different from the plug in FIG. 3. Furthermore, the initial position of the plugs beyond the bypass allows prefilling the syringe with a very limited amount of the two substances. The stroke of the syringe plunger being equal, a much smaller amount of medicinal fluid will be administered than with other single chamber syringes.

One of the prior arts which emerged in application WO2017216651 A1, i.e. European patent application EP0568321 A2, shows solutions similar to the above, in which the front plug must be pierced by a double tip syringe needle or a second bypass, which bypasses the front plug itself, is used. In the first case, the piercing operation is not sufficiently safe; in the second case, the construction of a second bypass is complicated. In both cases, structures and elements must be introduced which require a more articulated production line and therefore the product will be more expensive as a whole, while its operation will be far from optimal.

### SCOPE AND OBJECT OF THE INVENTION

It is the object of the present invention to solve the problems of the prior art by taking the needs of the sector into account.

In particular, it is the object of the present invention to provide a device for delivering pharmaceutical substances which allows an optimal operation by means of a simple and inexpensive system.

The present invention relates to a prefilled injection device according to one or more of the appended claims.

### DETAILED DESCRIPTION OF EXAMPLES OF PREFERRED EMBODIMENTS OF THE INVENTION

### List of Drawings

The invention will now be described by way of example, with particular reference to the drawings of the accompanying figures, in which:

FIG. 1 shows the different and progressive states of use of a double chamber syringe for the subsequent administration of two liquids, according to the prior art;

FIG. 2 shows a sliding plug used in the syringe of FIG. 1, according to the prior art;

FIG. 3 shows a double chamber syringe for the pre-reconstruction of a solution and subsequent administration of the solution, according to the known art;

FIG. 4 shows the different and progressive states of use of a double chamber syringe for the pre-reconstruction of a solution and subsequent administration of the solution, according to the present invention;

FIG. 5 shows the same plug as FIG. 2, this time used in the device of FIG. 4; and

FIG. 6 shows the same embodiment as FIG. 4, but with the liquids/powder in motion.

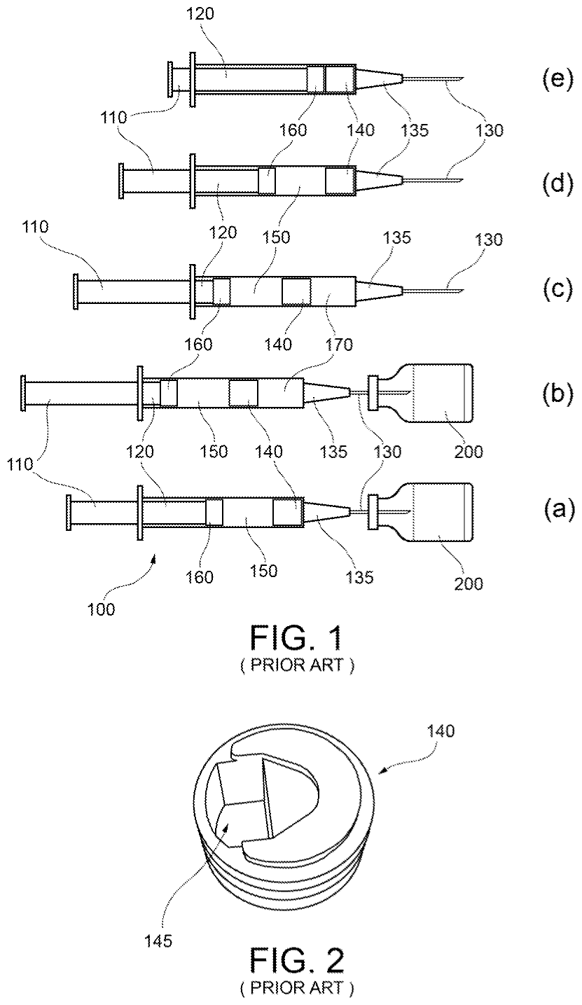

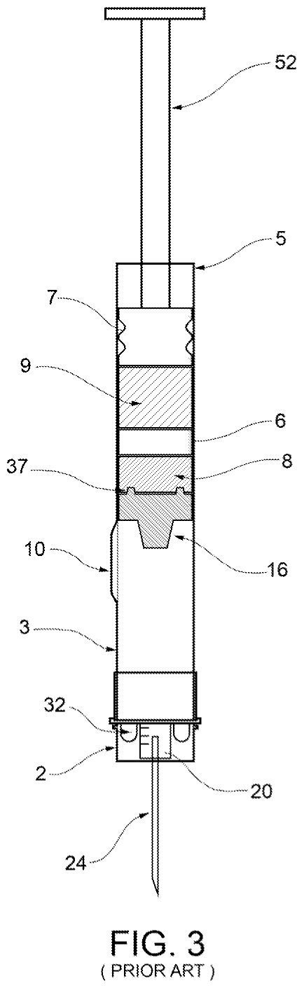

It is worth noting that hereinafter elements of different embodiments may be combined together to provide further embodiments without limitations respecting the technical concept of the invention, as a person skilled in the art will effortlessly understand from the description.

The present description also makes reference to the prior art for its implementation, with regard to the detail features which are not described, such as, for example, elements of minor importance usually used in the prior art in solutions of the same type.

When an element is introduced, it is always understood that there may be “at least one” or “one or more”.

When a list of elements or features are listed in this description, it is understood that the finding according to the invention “comprises” or alternatively “consists of” such elements.

### Embodiments

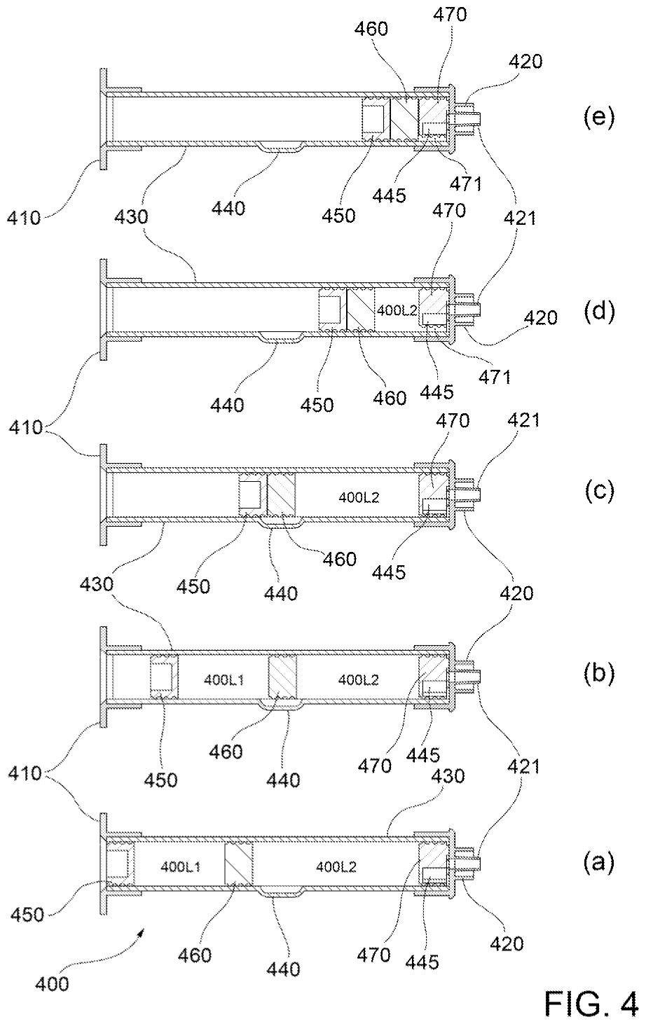

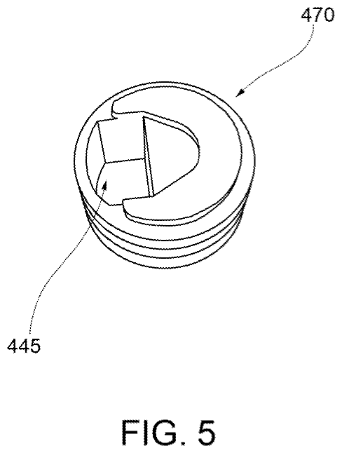

With reference to FIGS. 4 and 5, the prefilled injection device 400 comprises, according to an embodiment of the present description, a typical tubular containment body 430, which extends along an axis (not shown) between a front end element 420 and a rear end element 410. It is apparent that, as in all syringes, the front end element 420 is a partial closure element, with a spout 421 to which a needle or a small tube (not shown) is connected for administering or transferring the liquid or solution into the syringe.

Since this is a device in which the substances are already in the syringe and must be mixed, there are a first plug 470 and a second plug 460 arranged inside the tubular body 430 of the syringe, so as to delimit therebetween a second containment chamber 400L2 in the tubular body 430 itself containing a second liquid substance. At the same time, there is a third plug 450 connected to a syringe piston (not shown) and forming a first containment chamber 400L1 with said second plug 460 in the tubular body 430. The second containment chamber 400L2 contains a second substance, either solid or liquid, e.g. in powder form.

The second plug 460 hermetically separates the first containment chamber 400L1 from the second containment chamber 400L2. The second plug 460 and the third plug 450 are configured and adapted to slide inside the tubular body 430.

It is further apparent that, as in the traditional syringes of this type, there is at least one bypass channel 440, initially in a closed state and adapted to be taken into an opened state for mixing said first and said second substance. The bypass channel may be formed in various manners in the syringe.

According to an embodiment of the present description, and contrarily to the mentioned known art, in the prefilled injection device or syringe 400 the first plug 470 is arranged in a fixed position in contact with or near said front end element 420 along the axial direction. “Proximity” means a distance (either minimum or not) of construction convenience which can be easily determined according to the case.

It is worth noting here that the solution of application WO2017216651 A1 cannot use such a plug in fixed position, because such a device must store a predetermined amount of reconstituted liquid and must be able to move the plugs to allow the administration thereof. Furthermore, the bypass cannot be placed, in the initial state, between the two plugs, but both plugs must stay above the bypass, thereby limiting the amount of substances in the syringe.

By putting a fixed plug into abutment at the front end of the syringe as shown, a passage for the liquid is needed, and it is desirable that this does not complicate the constitution of the syringe itself.

This is achieved, according to an aspect of the present description, with a plug deformable under the pressure of the liquid which is pushed by the action of the plunger or piston of the syringe 400. In general, one-way passage means may be used, e.g. such as one-way valves. More in general, liquid communication means with said front end element 420 will be provided with/in said first plug.

According to a preferred embodiment, the first plug 470 is provided with a non-through cavity 445 which is offset with respect to said axis and facing towards said front end element 420 (the cavity 445 of 470 is facing towards 420, because if it were facing 410 it would be filled with solution, thereby making the plug stick on the walls of 430, because it is filled with liquid). This allows the plug to contract at least partially perpendicularly to said axis, and thus to create a channel 471 with the body 430 up to the spout 421 of the end element 420.

FIG. 5 shows an embodiment of the plug 470 with cavity 445, according to the invention.

The deformability, that is the narrowing of the plug 470 can be achieved by means of various inner configurations of the plug itself, e.g. by providing inner elements which can collapse or break (and in this case the parts which will break will remain advantageously coupled to the rest of the structure of the plug) under liquid pressure.

According to an embodiment of the present invention, the tubular body 430 comprises an inner wall provided with a recess adapted to define the bypass channel 440. In the initial configuration of the plugs, the bypass channel is positioned between the first plug 470 and the second plug 460.

According to a preferred aspect of the present description, the length of the bypass channel 440 is larger than the total length of the second plug 460, but smaller than the sum of the lengths of the plugs 450 and 460 (to prevent the liquid from flowing back once the third one 450 reaches the second one 460).

Alternatively, the bypass channel may be provided in the second plug 460, e.g. with a shrinkage following a collapse of the inner structure, or with an appropriate one-way valve (e.g. breakable membrane).

From the point of view of the materials of the syringe 400, according to the present description, the front end element 420 can be made of plastic material and/or made in one piece with the tubular body 430. Similarly, the rear end element 410 may be made of plastic material and/or made in one piece with the tubular body. The tubular body may be made of plastic or glass, for example.

From the point of view of the substances present from the beginning in the syringe, the first liquid substance is a solvent for injectable use and the second substance may be an active substance or a highly active substance, e.g. an antibiotic, or a beta-lactam antibiotic (Cephalosporin and/or Penicillin antibiotic), or a cytotoxic anticancer substance, or a hormone, or a biological preparation, or a biotechnological product, or a monoclonal antibody, or a protein, or a vaccine, or an anesthetic.

Turning to the steps of operation of the device according to the present description, FIG. 4(a) shows the starting state of the device as described above, in which the plunger is not shown for ease of representation. The plunger can be connected to the third plug 450.

In FIG. 4(b), the plunger starts moving the plug 450 and, by means of the liquid of the chamber 400L1, also the plug 460 towards the zone of the bypass 440. Again in FIG. 4(b), the bypass is activated because of the position of the plug 460 inside the bypass itself, thereby allowing the liquid to pass from 400L1 to 400L2.

In FIG. 4(c), once the bypass is activated because of the position of 460 inside the bypass itself, 450 and 460 end up touching each other, the liquid being passed either entirely or almost totally from chamber 400L1 to chamber 400L2, thereby reconstituting itself with the second substance.

At this point, again with further push of the piston and thus of the plug 450, the second chamber 400L2 is reduced to FIG. 4(d) and substantially or totally canceled in the final state of FIG. 4(e). This is possible by virtue of the fluid communication means comprised in or constituting the front plug 470, which allow the reconstituted substance to reach the spout 421 and from there a needle or a tube or other fluid connection to a third body (e.g. the body of a patient who is injected with the syringe of the present description).

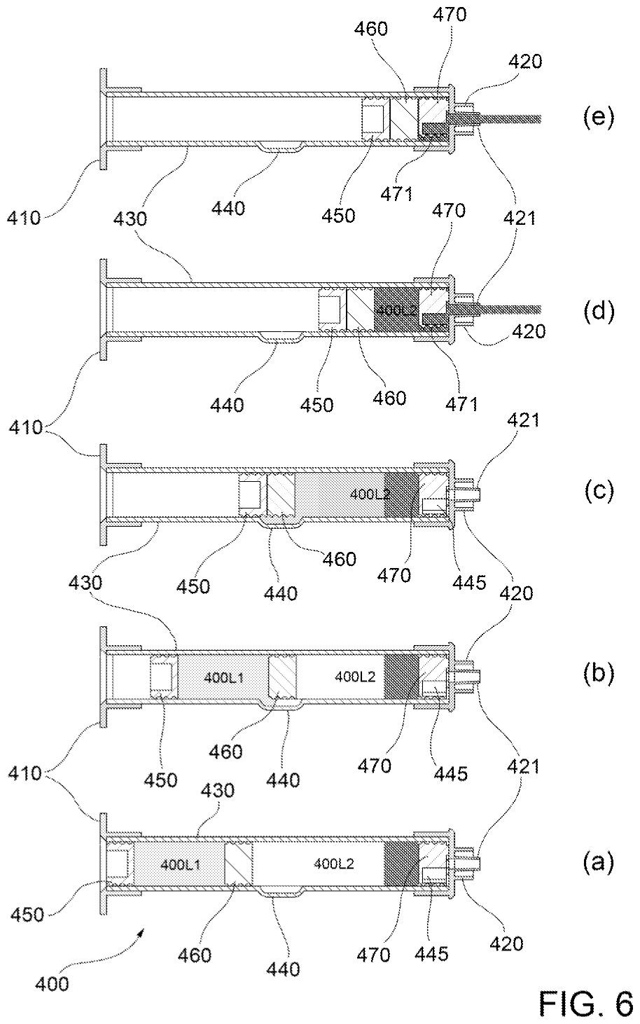

FIG. 6 shows the injection sequence as in FIG. 4 but with the various materials indicated in the syringe 400 with different patterns.

### Advantages of the Invention

As explained above, among the advantages obtained with the device of the present description, there is the simple structure of the syringe with maximization of the substances to be injected at the same time. The substances to be (reconstituted and) injected being equal, smaller syringes than those of the prior art can therefore be made.

For this reason, but also because of the general shape similar to that of the prior art, and for the simplicity of the characterizing elements, production costs are minimized and operation is optimal.

Hereto, we have described the preferred embodiments and suggested some variants of the present invention, but it is understood that a person skilled in the art can make modifications and changes without departing from the respective scope of protection, as defined by the appended claims.

1.-14. (canceled)

15. A prefilled injection device, comprising:
a tubular containment body, which extends along an axis between a front end element and a rear end element, the front end element comprising a spout, a needle being connectable to said spout;
a first plug and a second plug arranged inside the tubular containment body, so as to delimit therebetween a second containment chamber in the tubular containment body, the second containment chamber containing a second solid or liquid substance;
a third plug connected to a syringe piston and forming a first containment chamber with said second plug in the tubular containment body, the first containment chamber containing a first liquid substance; and
at least one bypass channel, configured to assume alternatively a closed state or an opened state;
wherein:
the first plug is arranged in a fixed position either near or in contact with said front end element along said axis;
the second plug hermetically separates the first containment chamber from the second containment chamber;
the second and third plugs are configured to slide inside the tubular body;
the tubular containment body comprises an inner wall provided with a recess defining said at least one bypass channel, wherein the at least one bypass channel is positioned between the first plug and the second plug in an initial configuration of the first and second plugs; and
said first plug comprises liquid communication means for passing, upon a movement of the syringe piston towards the front end element, from a first closed state to a second opened state.

16. The prefilled injection device of claim 15, wherein a length of the at least one bypass channel is larger than a total length of the second plug but smaller than the sum of the lengths of the third and second plugs.

17. The prefilled injection device of claim 15, wherein said liquid communication means are provided in the structure of said first plug, wherein the liquid communication means in the first plug comprise a watertight closure with respect to said front end element in said first closed state and said first plug is shrunk perpendicularly to said axis in said second opened state, thereby forming a liquid communication channel between said second containment chamber and said front end element.

18. The prefilled injection device of claim 17, wherein said liquid communication means in the first plug comprise a non-through cavity which is offset with respect to said axis and facing towards said front end element.

19. The prefilled injection device of claim 15, wherein the front end element is made of plastic material.

20. The prefilled injection device of claim 15, wherein the front end element is made in one piece with the tubular body.

21. The prefilled injection device of claim 15, wherein the rear end element is made of plastic material.

22. The prefilled injection device of claim 15, wherein the rear end element is made in one piece with the tubular body.

23. The prefilled injection device of claim 15, wherein the tubular body is made of plastic or glass material.

24. The prefilled injection device of claim 15, wherein the first liquid substance is a solvent for injectable use and the second substance is an active substance or one of an antibiotic, a beta-lactam antibiotic, Cephalosporin, Penicillin antibiotic, a cytotoxic anticancer substance, a hormone, a biological preparation, a biotechnological product, a monoclonal antibody, a protein, a vaccine, and an anesthetic.

25. A prefilled injection device, comprising:
a tubular containment body, which extends along an axis between a front end element and a rear end element, the front end element comprising a spout, a needle being connectable to said spout;
a first plug and a second plug arranged inside the tubular containment body, so as to delimit therebetween a second containment chamber in the tubular containment body, the second containment chamber containing a second solid or liquid substance;
a third plug connected to a syringe piston and forming a first containment chamber with said second plug in the tubular containment body, the first containment chamber containing a first liquid substance; and
at least one bypass channel, configured to assume alternatively a closed state or an opened state;
wherein:
the first plug is arranged in a fixed position either near or in contact with said front end element along said axis;
the second plug hermetically separates the first containment chamber from the second containment chamber;
the second and third plugs are configured to slide inside the tubular containment body;
the at least one bypass channel is provided in the second plug; and
said first plug comprises liquid communication means for passing, upon a movement of the syringe piston towards the front end element, from a first closed state to a second opened state.

26. The prefilled injection device of claim 25, wherein the at least one bypass channel consists of a one-way valve integrated in said second plug.

27. The prefilled injection device of claim 26, wherein the at least one bypass channel consists of inner elements of the second plug which are collapsible or breakable under liquid pressure.

28. The prefilled injection device of claim 25, wherein said liquid communication means are provided in the structure of said first plug, wherein the liquid communication means in the first plug comprise a watertight closure with respect to said front end element in said first closed state and said first plug is shrunk perpendicularly to said axis in said second opened state, thereby forming a liquid communication channel between said second containment chamber and said front end element.

29. The prefilled injection device of claim 28, wherein said liquid communication means in the first plug comprise a non-through cavity which is offset with respect to said axis and facing towards said front end element.

30. The prefilled injection device of claim 25, wherein the front end element is made of plastic material.

31. The prefilled injection device of claim 25, wherein the front end element is made in one piece with the tubular body.

32. The prefilled injection device of claim 25, wherein the rear end element is made of plastic material.

33. The prefilled injection device of claim 25, wherein the rear end element is made in one piece with the tubular body.

34. The prefilled injection device of claim 25, wherein the tubular body is made of plastic or glass material.

35. The prefilled injection device of claim 25, wherein the first liquid substance is a solvent for injectable use and the second substance is an active substance or one of an antibiotic, a beta-lactam antibiotic, Cephalosporin, Penicillin antibiotic, a cytotoxic anticancer substance, a hormone, a biological preparation, a biotechnological product, a monoclonal antibody, a protein, a vaccine, and an anesthetic.
